(19) United States
(12) Patent Application Publication
(10) Pub. No.: US 2026/0041334 A1
(43) Pub. Date: Feb. 12, 2026

(54) SELF-SUPERVISED LEARNING AND OPPORTUNISTIC INFERENCE FOR CONTINUOUS MONITORING OF FREEZING OF GAIT IN PARKINSON'S DISEASE

(71) Applicant: ARIZONA BOARD OF REGENTS ON BEHALF OF ARIZONA STATE UNIVERSITY, Scottsdale, AZ (US)

(72) Inventors: Hassan Ghasem Zadeh, Phoenix, AZ (US); Shovito Barua Soumma, Phoenix, AZ (US)

(21) Appl. No.: 19/295,312

(22) Filed: Aug. 8, 2025

Related U.S. Application Data
(60) Provisional application No. 63/681,752, filed on Aug. 9, 2024.

(51) Int. Cl. A61B 5/11; G06N 3/0455; G06N 3/082; G16H 50/30

(52) U.S. Cl. CPC A61B 5/112; G06N 3/0455; G06N 3/082; G16H 50/30

(57) ABSTRACT

A method is described herein comprising receiving first accelerometer data of a plurality of subjects, processing the first accelerometer data to generate a first dataset and a second dataset, wherein the first dataset omits labeling information of freeze of gait events, wherein the second dataset includes labeling information of freeze of gait events, training an encoder block using the first dataset, generating a classification model using the trained encoder and the second dataset, and applying the classification model to second accelerometer data of a subject to identify a health state of the subject.

### RELATED APPLICATIONS

This application claims priority to U.S. Application No. 63/681,752, filed Aug. 9, 2025.

### GOVERNMENT INTERESTS

This invention was made with government support under 2227002 awarded by the National Science Foundation. The government has certain rights in the invention.

### TECHNICAL FIELD

The disclosure herein involves systems and methods for detecting freezing of gait in Parkinson's Disease.

### BACKGROUND

Parkinson's disease (PD) is a progressive neurological disorder affecting 7-10 million people worldwide, significantly impacting their quality of life. Freezing of Gait (FoG), a transient inability to produce effective steps during walking [1], is one of the most debilitating symptoms of PD, leading to poor mobility, increased risk of falls and injuries, and reduced quality of life. While treatments like levodopa can sometimes reduce the severity of freezing episodes, their effectiveness is often incomplete and variable, diminishing over time [2]. Compensatory treatments such as on-demand cueing require patient or companion initiation, which can be challenging in time-sensitive or anxiety-provoking situations that trigger freezing [3]. Identifying FoG events and, more importantly, the time leading up to a FOG event could result in early deployment of on-demand cues to help reduce the severity of a freezing event [4].

### INCORPORATION BY REFERENCE

Each patent, patent application, and/or publication mentioned in this specification is herein incorporated by reference in its entirety to the same extent as if each individual patent, patent application, and/or publication was specifically and individually indicated to be incorporated by reference.

### SUMMARY OF THE INVENTION

A method is described herein under an embodiment comprising receiving first accelerometer data of a plurality of subjects, processing the first accelerometer data to generate a first dataset and a second dataset, wherein the first dataset omits labeling information of freeze of gait events, wherein the second dataset includes labeling information of freeze of gait events, training an encoder block using the first dataset, generating a classification model using the trained encoder and the second dataset, and applying the classification model to second accelerometer data of a subject to identify a health state of the subject.

In embodiments, the training the encoder block comprises masking fixed length segments of the first dataset to produce a third dataset.

In embodiments, the training the encoder block comprises producing a lower dimensional representation of the third dataset.

In embodiments, the training the encoder block comprises passing the lower dimensional representation through a fully connected neural network to generate encoder block weights for predicting the masked fix length segments.

In embodiments, the generating the classification model comprises adding additional neural network layers to the trained encoder.

In embodiments, the generating the classification model comprises training the additional layers using the second dataset and while freezing weights of the pretrained encoder.

In embodiments, the applying the classification model includes monitoring a magnitude of the second accelerometer data.

In embodiments, the applying the classification model comprises activating the classification model when a magnitude of the second accelerometer data exceeds a threshold value.

In embodiments, the processing the first accelerometer data includes balancing freeze of gait and non freeze of gait portions of the training data.

In embodiments, the balancing comprises applying a windowing overlap to freeze of gait and non freeze of gait portions of the accelerometer data.

In embodiments, the windowing overlap includes a fifty percent overlap for non field of gate periods.

In embodiments, the windowing overlap includes a seventy five percent overlap for field of gate periods.

In embodiments, the state comprises a presence or absence of a freeze of gate event.

A system is described herein comprising one or more applications running on a server, the one or more applications for providing receiving first accelerometer data of a plurality of subjects, processing the first accelerometer data to generate a first dataset and a second dataset, wherein the first dataset omits labeling information of freeze of gait events, wherein the second dataset includes labeling information of freeze of gait events, training an encoder block using the first dataset, generating a classification model using the trained encoder and the second dataset, the one or more applications providing the classification model to a wearable device as a mobile application, wherein the mobile application runs on an a processor of the wearable device, wherein the wearable device monitors second accelerometer data of a wearable device user, wherein the mobile application applies the classification model to the second accelerometer data of the user to identify and notify the user of a freeze of gate event, wherein the applying the classification model includes monitoring a magnitude of the second accelerometer data and activating the classification model when a magnitude of the second accelerometer data exceeds a threshold value.

In embodiments, the generating the classification model comprises adding additional neural network layers to the trained encoder.

In embodiments, the generating the classification model comprises training the additional layers using the second dataset and while freezing weights of the pretrained encoder.

In embodiments, the processing the first accelerometer data includes balancing freeze of gait and non freeze of gait portions of the training data.

In embodiments, the balancing comprises applying a windowing overlap to freeze of gait and non freeze of gait portions of the accelerometer data.

### BRIEF DESCRIPTION OF THE DRAWINGS

FIG. 1 shows a self-supervised training pipeline for real-time FoG detection, under an embodiment.

FIG. 2 shows an activity threshold-based triggering mechanism for computational offloading and battery life extension in wearable FoG detection system, under an embodiment.

FIG. 3A illustrates DHWT segmentation process for training set, under an embodiment.

FIG. 3B shows FoG proportion using standard vs. DHWT segmentation, under an embodiment.

FIG. 4 shows an architecture of a stacked 1D CNN model. Input to the model is 3-minute raw sensor data, under an embodiment.

FIG. 5 shows A: FoG Episode, B: False Positive, C: Normal Episode, D: Detected FoG Episode (TP), E: Detected Normal Episode (TN), and L: Latency, under an embodiment.

FIG. 6 shows Receiver Operating Characteristic (ROC) curves for supervised and self-supervised models, under an embodiment.

FIG. 7 shows impact of activity threshold on SSL performance, under an embodiment.

FIG. 8 shows effect of activity threshold on inference time, under an embodiment.

FIG. 9 illustrates the Impact of window size on FoG detection latency across different episode durations (Small, Medium, Large), under an embodiment.

FIG. 10 shows performance analysis for different amounts of labeled data, under an embodiment.

### DETAILED DESCRIPTION

### I. Introduction

In recent years, wearable technology and machine learning (ML) algorithms have emerged as promising tools for continuous monitoring and management of PD symptoms, including gait disturbances and tremors [5]-[7] However, real-world deployment of these technologies for continuous monitoring and intervention has remained a significant research challenge. One major hurdle is the scarcity of labeled data essential for training robust ML models, particularly in PD, where symptom manifestation is highly individualized. Data annotation requires considerable time and expertise, limiting the availability of accurate and diverse datasets [8].

Detecting FoG using a patient-independent model is a complex task due to significant inter- and intra-variability in patients' gait patterns. Previous research on FoG detection relied on multiple sensors, extensive feature engineering, patient-specific data collection, and model retraining, limiting large scale adoption of wearable technologies for long-term health monitoring [9]-[11]. These challenges are addressed using an innovative label-efficient, patient-independent, and robust self-supervised learning framework, LIFT-PD (Label-efficient In-home Freezing-of-gait Tracking in Parkinson's Disease), for detecting FoG events in real-time. The main contributions of this work are:
1) Label-Efficient Learning: LIFT-PD uses a self-supervised learning approach, allowing the model to learn from unlabeled data with minimal labeled instances required for fine-tuning. This significantly reduces the need for extensive data annotation, making it more practical for real-world deployment where labeled data is often limited.
2) Handling Variability and Imbalanced Data: A Differential Hopping Windowing Technique (DHWT) effectively handles inter and intra-patient gait variations and data imbalance issues.

TABLE I
Summary of Recent FoG Detection Works.

| | | Sensor (Number) | Preprocessing/ | | |
|---|---|---|---|---|---|
| Study | Method | (Position) | Extracted Features | Validation | Contribution |
| [9] | CNN and Transformer | Accelerometer (1) | Resample, Filtering, | LOSO CV (SI) | Transformer and CNN based methodologies |
| | (Supervised) | (Left Waist) | FFT, FI | | for FoG detection, along with a clustering |
| | | | | | approach for FoG episode analysis |
| [12] | Multihead CNN | | X | Hold Out (SI) | Light algorithm for real time FoG detection |
| | (Supervised) | | | | using raw sensors data. |
| [5] | Supervised ML | IMU (2) | Time & Frequency | 10 Fold CV LOSO | Detect and predict FoG considering the impact |
| | | (Shin) | Domain Features | Train-test 70-30% | of dopaminergic therapy on performance. |
| [11] | Semi Supervised | IMU (1) | Filtering, 3 Features | LOSO (SD) | Adapting FoG classifier's parameters in real- |
| | 3 Layers | (Ankle) | (FI, SP, STD) | | time with unlabeled data |
| [10] | Supervised, | IMU (2) | Filtering, | LOOCV (S( ) | One-class classifier for FoG detection using |
| | Transfer learning | (Left-Right Ankle) | Data Augmentation | | only normal gait data |

FI: Freezing Index,
SP: Stride Peak,
STD: Standard Deviation,
SD: Subject Dependent,
SI: Subject Independent,
LOOCV: Leave-One-Out Cross-Validation

3) Robust Performance: Despite using significantly fewer labeled data points, LIFT-PD achieves comparable performance to fully supervised approaches.
4) Energy Efficiency: LIFT-PD includes an opportunistic inference module that activates the model only during active movement periods, significantly reducing computational load and power consumption, paving the way for implementation in a stand-alone wearable system for real-time symptom monitoring.
5) Practical Deployment: By using a single triaxial accelerometer and processing data in real-time without extensive preprocessing, LIFT-PD offers a practical, scalable solution for continuous, in-home real-time monitoring of PD patients, facilitating seamless integration into everyday healthcare systems.

These contributions make LIFT-PD a scalable, energy-efficient, and patient-independent solution for real-time FoG detection, with significant potential for improving Parkinson's disease management through continuous and accessible monitoring.

### II. Related Work

The detection of FoG events in PD has been the subject of extensive research, often involving multimodal datasets. In particular, prior work explored the effectiveness of different sensor modalities such as gait acceleration, electroencephalogram (EEG), electromyography (EMG), and skin conductance (SC) in FoG detections [13]. Furthermore, researchers used an LSTM-based model combining acceleration and synthetic EEG data [14]. Previous work also introduced transfer learning [15], data augmentation technique and resampling, which are efficient in scenarios with limited labeled data [16]. One can broadly categorize these prior works based on label usage into supervised learning approaches for motion analysis and weakly labeled approaches, including semi-supervised and self-supervised learning, for FoG detection.

Supervised methods for FoG detection often rely on large amounts of labeled data, which is a significant limitation in real-world applications. These methods typically use machine learning (ML) algorithms such as CNNs, transformers, and LSTMs [14] combined with sensor modalities like accelerometers and IMUs. Multi-head CNN [12] and CNN-Transformer models [9] have shown good performance in detecting FoG in PD patients, but they face challenges in generalization and high computational requirements, which limit their feasibility for real-time wearable monitoring. While these models provide high detection rates, their reliance on extensive feature engineering [5], [17], a large number of training samples, and significant computational resources makes them impractical for wearable, real-time monitoring. Additionally, the models struggle with high false-positive rates, which can reduce their effectiveness in real-world settings where minimizing such errors is crucial for patient safety and usability. Transfer learning techniques, such as those used in the One-Class Classifier [10], help improve detection in normal conditions but fail to adapt to the dynamic and complex nature of FoG episodes, limiting their effectiveness in real-world applications. Table I summarizes recent PD studies on FoG detection. To address the challenge of insufficient labeled data, some studies have turned to semi-supervised learning (semi-SL), using a combination of labeled and unlabeled data for model training. Mikos et al. [11] uses a three-layer network with an IMU at the ankle, incorporating filtering and feature extraction. While the approach shows improvement over purely supervised models, it still requires a considerable amount of labeled data and suffers from issues related to the inability to generalize effectively across different PD patient profile.

The application of self-supervised learning (SSL) to timeseries data, particularly for human activity recognition (HAR), is a newer area of research. Unlike supervised and semisupervised methods, SSL frameworks like SimCLR [18] focus on learning representations without relying on labeled data, making them ideal for scenarios with limited annotations. Originally, it was designed for computer vision tasks, but was later adapted for HAR using a transformer-based encoder method [19]. However, the use of SSL for FoG detection remains limited due to challenges in effectively balancing the dataset and ensuring model generalization to diverse populations. The efficacy of self-supervised learning in medical time series analysis has been demonstrated, emphasizing its role in data augmentation and contrastive pair formation [20].

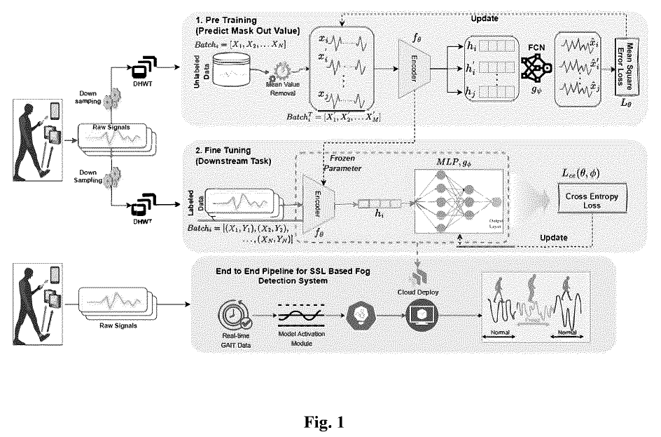

FIG. 1 illustrates a self-Supervised training pipeline for real-time FoG detection. Pretrain: Encoder reconstructs masked signal segments, outputting predicted values hᵢ for missing data. Fine-tuning: Encoder weights are frozen while the MLP is optimized using cross-entropy loss on labeled data. End-to-End Pipeline: The trained model is integrated into a real-time system, where model activation module (MAM) selectively activates the computationally intensive FoG detection model for energy-efficient, long-term monitoring.

While numerous studies have proposed effective methods for FoG detection, most rely on either extensive labeled data or computationally expensive models that are unsuitable for real-time, wearable deployment. These methods often depend on data collected in controlled environments, limiting their practical utility for everyday monitoring. Additionally, existing approaches struggle with imbalanced datasets, where FoG events are underrepresented and have limited generalization across diverse patient profiles, such as varying severities, ages, and genders. In contrast, LIFT-PD introduces a self-supervised framework that addresses these challenges by leveraging a Differential Hopping Windowing Technique (DHWT) for handling class imbalances during training. By using a minimal amount of labeled data and combining Opportunistic Inference Modules to optimize power consumption, LIFT-PD achieves real-time FoG detection with significantly reduced computational overhead. LIFT-PD's use of a single triaxial accelerometer and real-time data processing without extensive preprocessing makes it highly practical for wearable devices, unlike traditional models that rely on multiple sensors and are too resource-intensive for continuous monitoring.

### III. Lift-PD System Design

Training robust deep learning (DL) models typically requires large amounts of labeled data, which can be challenging to obtain, especially for tasks like freezing of gait (FoG) detection in elderly. To address this issue, LIFT-PD employs self-supervised learning to utilize unlabeled data during training. Another challenge in designing ML models for FoG detection is that FoG events are sparse, resulting in an imbalanced dataset with a significantly lower proportion of FoG episodes compared to non-FoG activities. To mitigate this challenge, the LIFT-PD approach incorporate the ‘Differential Hopping Windowing Technique (DHWT)’ during data preprocessing, which applies variable overlaps for FoG and non-FoG instances, thereby enhancing the model's ability to learn from the underrepresented class. Finally, an opportunistic-based lightweight algorithm is introduced to reduce execution complexity, allowing for implementation in stand-alone wearable devices (Section III-B).

### A. Problem Modeling by Self-Supervised Learning (SSL)

The FoG event detection problem is framed as a multivariate time-series classification task. At each time stamp t, the input raw signal is represented as a vector

$${x}_{t} = [ {x}_{t}^{1} , {x}_{t}^{2} , {x}_{t}^{3} ]$$

where xₜ∈ [symbol] ^{C=3} and C corresponds to the three-channel (x, y, z) accelerometer data. These raw signals are then combined into a matrix, X=[x₁, x₂, . . . , x_{T}]∈ [symbol] ^{T×C}. After applying the DHWT method, the signals are transformed into N number of training frames (windows) (X∈ [symbol] ^{T×C}→X_{W}∈ [symbol] ^{N×T′×C}) where x_{w}_{ᵢ}∈X_{W} represents i^{th} window, T′ is the windowing time length and X_{w}=[x_{w}_{₁}, x_{W}_{₂}, . . . , x_{W}_{_{N}}].

$$D : X ∈ {ℝ}^{T \times C} \to {X}_{W} ∈ {ℝ}^{N \times {T}^{′} \times C}$$

$${X}_{w} = [ {x}_{{w}_{1}} , {x}_{{w}_{2}} , … , {x}_{{w}_{N}} ]$$

The ultimate goal is to correctly assign a label y∈{0, 1} to each window, where y=1 represents “FoG” and y=0 represents “non-FoG”.

LIFT-PD uses SSL for FoG detection in two steps: learning contextual representations from raw signals using a 1D CNN model, followed by performing the downstream FoG detection task. In this paper, raw accelerometer signals are used as physiological signals and the downstream task is binary ‘Freezing of Gait’ detection. In the following subsections, the two main components of an SSL approach are described:
1) Pretext Task (Signal Reconstruction): During pretraining, a 3-layer neural network model with an encoder is trained using SSL. Random segments of fixed length m are masked from each window, x_{w}_{ᵢ} by replacing them with zeros. The model is then trained to predict these masked values. as shown in FIG. 1 (Part 1). This whole training process does not need any labeled data. The masked signal data \hat{X}_{w}∈ [symbol] ^{N×T′}, is defined by \hat{X}_{w}=mask(X_{w},m) where \hat{x}_{w}_{ᵢ}∈\hat{X}_{w} The encoder ƒ_{θ}: [symbol] ^{N×T′}→ [symbol] ^{N×D} reconstructs the original signal from the masked windows, producing a lower dimensional hidden representation H∈ [symbol] ^{N×D}, where hᵢ∈ [symbol] ^{D}↓ƒ_{θ}(\hat{x}_{w}_{ᵢ}). These are passed through a fully connected network g_{ψ}(⋅) to predict the masked segments represented as \hat{x}=g_{ψ}(hᵢ). The encoder minimizes the reconstruction loss Le using mean squared error (MSE) defined as

$${L}_{θ} = \frac{1}{{N}_{m}} {\sum }_{j = 1}^{{N}_{m}} {( {\hat{x}}_{j} - {x}_{p ( j )} )}^{2} ,$$

where Nₘ is the total number of masked points, \hat{x}ⱼ is the prediction for the j-th masked point, and xₚ₍ⱼ₎ is the original input value, whose position is p(j) in the segmented signal windows.
2) Downstream Task (FoG Classification): After pretraining, a Multilayer Perceptron (MLP) g_{ψ}: [symbol] ^{N×D}→Y, is introduced on top of the encoder for binary FoG detection. The MLP maps the learned embeddings H to FoG or non-FoG labels Y, represented as Ŷ=g_{ϕ}(H)=g_{ϕ}(ƒ_{θ}(X_{w})), where ƒ_{θ} is the pre-trained encoder with parameters θ.

The fine-tuning step is supervised, using a small amount of labeled data, with the encoder weights frozen and only the MLP layers trained with a lower learning rate as shown in FIG. 1 (Part 2). The LIFT-PD approach minimizes the binary cross-entropy loss L_{ce}(θ,ϕ) for the downstream task defined as

$${L}_{ce} ( θ , ϕ ) = - \frac{1}{N} {\sum }_{i = 1}^{N} [ {y}_{i} log ( σ ( {\hat{y}}_{i} ) ) + ( 1 - {y}_{i} ) log ( 1 - σ ( {\hat{y}}_{i} ) ) ] ,$$

where N is the total number of windows, y is an indicator variable (1 for FoG, 0 for non-FoG), σ is the sigmoid function, and ŷ∈Ŷ is the output of the classifier g_{ϕ}(hᵢ).

### B. Opportunistic Inference

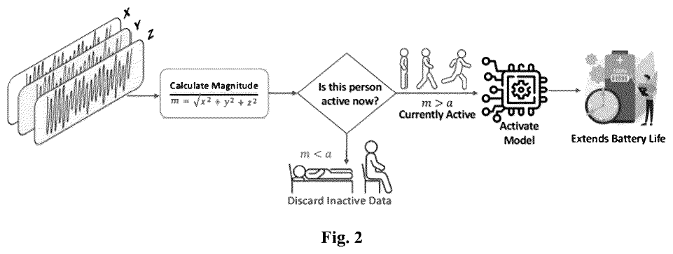

To optimize power consumption and computational resources for real-life in-home PD monitoring using wearable devices, LIFT-PD implemented an opportunistic-based naive algorithm as a model activation module (MAM). This module differentiates between active and inactive periods, activating the computationally heavy self-supervised learning (SSL) FoG detection model only during identified active intervals, as shown in FIG. 2. The activation module filters out inactive windows, ensuring the SSL model is selectively executed only when activity is detected, significantly reducing power consumption and avoiding false positives during inactivity. During inactive periods, simpler methods handle the data by comparing the magnitude of the current incoming signal, leading to more efficient power utilization. For each window i∈[1,N], the magnitude Mᵢ of the 3D accelerometer is calculated as

$${M}_{i} = \frac{1}{{T}^{′}} {\sum }_{t = 1}^{{T}^{′}} ‖ \sqrt{{a}_{x , t}^{2} + {a}_{y , t}^{2} + {a}_{z , t}^{2}} ‖$$

where aₓ, a_{y}, and a_{z} are the acceleration signal along each axis. A window is considered active if Mᵢ≥α, where α is a predefined threshold (Eq. 1), otherwise MAM discards it.

$$\{ \begin{matrix}1 ( active ) & if {M}_{i} \geq α \\ 0 ( inactive ) & otherwise\end{matrix} \qquad ( 1 )$$

FIG. 2 illustrates a Model Activation Module: Activity threshold-based triggering mechanism for computational offloading and battery life extension in wearable FoG detection system.

The threshold was chosen to discard windows without degrading SSL algorithm performance. Finally, the effect of the magnitude threshold was evaluated separately during the inference time (Sec. IV-F).

### C. Imbalanced Training Data Mitigation

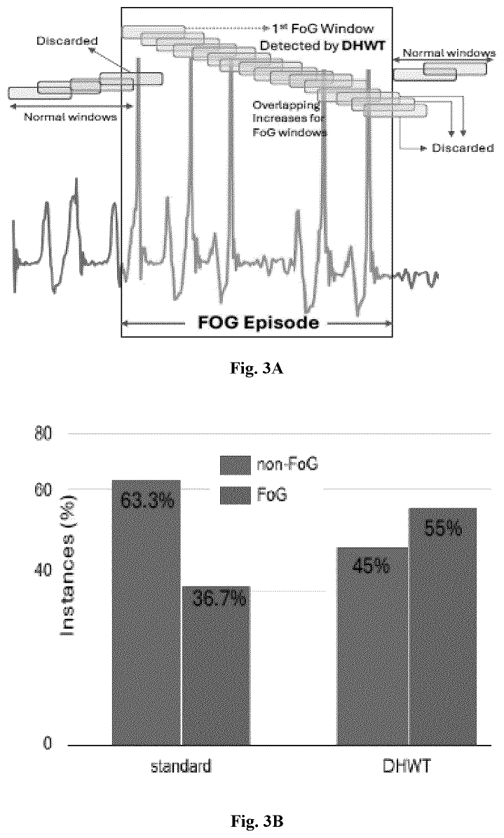

A proposed dataset generation method (DHWT) effectively handles imbalanced datasets without the need for additional preprocessing, balancing informative features with computational efficiency for real-time deployment on wearable devices. Raw sensor data are segmented into short, overlapping windows, adjusting the overlap based on activity type. For instance, in an experimental evaluation shown in FIG. 3A, this approach applied a 50% overlap for non-FoG periods and a 75% overlap for FoG episodes during training. During inference, windows are segmented using standard, non-variable overlaps to simulate real-world conditions where FoG episodes are not pre-identified. Using fixed-length overlaps typically results in an unbalanced training set, as illustrated in the left bar of FIG. 3B, with 63.3% non-FOG and 36.7% FOG instances. In contrast, the DHWT segmentation approach achieves a more balanced distribution, as shown in the right bar of the same figure, with non-FOG and FOG data at 45% and 55%, respectively. For test set generation, standard segmentation with a fixed 50% overlap (advancing every 1.5 seconds) mimics actual operating conditions, processing data from the preceding 3 seconds. A detailed explanation of the choice of a 3-second window length is provided in Section IV-G.

### D. Dataset

The dataset for this study is the publicly available tDCS FoG dataset, comprising movement data from PD subjects in both medicated (‘On’) and unmedicated (‘Off’) states during Freezing of Gait (FoG) provocation protocols [21]. Data were collected using a 3D accelerometer attached to the lower back, recording at 128 Hz, resulting in 1132 FoG episodes (285 minutes total) and 15.3 hours of recording. Each FoG episode was videotaped and annotated by experts [21]. The demographic and clinical characteristics of the subjects are summarized in Table II. The dataset contains events labeled as “Normal” or “Freezing of Gait” (FoG). The distribution of these events is summarized in Table III. The sessions followed the FoG provocation protocol as described in seminal studies by Reches et al. [22] and Manor et al. [23], and originally defined by Ziegler et al. [24].

FIG. 3A illustrates DHWT segmentation process for training set FIG. 3B shows FoG proportion using standard vs. DHWT segmentation.

TABLE II
Summary of patient characteristics

| | | | Medication | | | |
|---|---|---|---|---|---|---|
| Characteristics | Overall | | On | | Off | |
| Male (Female) | 8 | (32) | 7 | (31) | 6 | (20) |
| Age, mean (SD) | 69.5 | (7.75) | 70.9 | (6.5) | 68 | (8.3) |

| | | | | | |
|---|---|---|---|---|---|
| UPDRS ON, | 34.27 | (12.7) | 34.27 | (12.7) | — |
| mean (SD) | | | | | |

| | | | | | |
|---|---|---|---|---|---|
| UPDRS OFF, | 42.88 | (12.99) | — | 42.88 | (12.99) |
| mean (STD) | | | | | |

| | | | | |
|---|---|---|---|---|
| NFOGQ, mean (STD) | 17.12 | (7.57) | — | — |

| Years Since DX | 10.5 (5.9), [1, 23] |
|---|---|
| mean (SD), | |
| [min, max] | |

TABLE III
Event distribution during FoG provocation trials

| | Medication (%) | | |
|---|---|---|---|
| Event Type | On | Off | Total (%) |
| Normal | 28.01 | 35.41 | 63.42 |
| FoG | 18.73 | 17.86 | 36.59 |

The data was resampled to 40 Hz, which is considered an effective frequency for recognizing human activity through accelerometers in both healthy individuals and PD patients, including those experiencing Freezing of Gait (FoG) episodes. This frequency captures the typical freeze band (3-8 Hz) while reducing memory load and computational complexity.

### E. Mean Value Removal and Labeling

After segmentation, perform minimal pre-processing is performed on each window by removing the mean value from each of the sensor axes (e.g., x, y, z). This centering of the data mitigates sensor bias and reduces computational complexity. Finally, labels are assigned to the windows based on their content: non-Fog for windows containing only non-FoG data, and FoG for windows with at least 50% FoG data. Any windows containing a mix of both activities are discarded to ensure clear classification during the machine learning stage. This minimal preprocessing approach prioritizes real-time performance while preserving features relevant for FoG detection.

Algorithm 1: Activity Threshold Optimization
Algorithm for Prolonged Battery Life

| | |
|---|---|
| Input: | X: Set of inference data frames, |
| | [αₛₜₐᵣₜ, α_{final}]: Initial and final threshold, |
| | P₀: Baseline performance metric, |

f_{(.)}: Inference Model,

| | |
|---|---|
| | A_{(.)}: returns magnitude for xᵢ ∈ X |
| Output: | αₒₚₜ : Optimal activity threshold, |
| | Xₒₚₜ: Active frames for (αₒₚₜ) |

Initialization α ← αₛₜₐᵣₜ
while α ≤ α_{final} do

| \| | X_{active} = {xᵢ ∈ X \| A(xᵢ) ≥ α} | [symbol] Set of active |
|---|---|---|
| \| | frames; | |
| \| | N_{α} + \|X_{active}\| | [symbol] Size of active frames; |

| | |
|---|---|
| \| | P + f(X_{α}) |
| \| | if \|P − P₀\| ≤ θ then |
| \| | α ← α + δα |
| \| | else |
| \| | \| α_{opt ← α}; |
| \| | Xₒₚₜ = {xᵢ ∈ X \| A(xᵢ) ≥ αₒₚₜ}; |
| \| | break; |
| \| | end |

end
return αₒₚₜ, Xₒₚₜ

### F. Leave One Group Out (LOGO)

In order to evaluate the subject-independence, a leave-one-group-out cross-validation is preformed. The whole tdcs dataset is divided into two groups, each of them consisting of randomly chosen 20 patients. The two groups are generated so that patients in each group have similar characteristics in terms of age, duration of symptoms, and UPDRS. Hence, the patient-independent model is trained using 20 patients from each group using SSL, while the left-out group is stripped of its labels for validation. The classification performance for the left-out groups of patients is then evaluated and compared with the baseline supervised model. This procedure is repeated for each group within the dataset, conducted three times, and the results are averaged to ensure reliability and robustness in the findings.

### G. Model Development & Experimental Setup

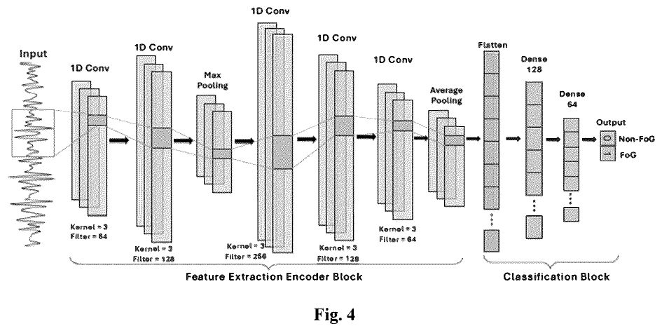

With respect to the downstream task, the method described herein implements a 5-layer 1D Convolutional Neural Network (CNN) architecture in LIFT-PD (FIG. 4), allowing us to use raw sensor data without extensive feature engineering. The model consists of an encoder block for feature extraction and a classification block for FoG detection. The encoder block has five 1D convolutional layers (filters: 64, 128, 256, 128, 64; kernel size: 3; ReLU activation) with max-pooling (pool size: 2) after the second layer and global average pooling (pool size: 2) after the fifth layer to downsample feature maps, reducing complexity. The flattened output is passed through an MLP with two dense layers (units: 128, 64; ReLU activation; dropout: 0.4 after the first layer) for binary classification, with a final dense layer (unit: 1; sigmoid activation) providing the FoG detection output.

FIG. 4 illustrates the Architecture of a stacked 1D CNN model. Input to the model is 3-minute raw sensor data.

The model was trained in two phases: pre-training and finetuning. In the pre-training phase, the encoder was trained for 70 epochs with a batch size of 64, using the Adam optimizer set to a learning rate of 0.01 and a decay of 0.001. During the fine-tuning phase, the additional dense layers were randomly initialized. The model was fine-tuned on labeled data for 40 epochs, maintaining the same batch size but with a reduced learning rate of 0.0001 for the classification task. The use of a lower learning rate (0.0001) during the fine-tuning phase is a common practice when working with pre-trained models. This lower learning rate helps preserve the learned features from the pre-training phase and ensures gradual adjustments, avoiding the drastic loss of previously learned representations.

All experiments (pre and post-processing) were performed on a computer with an Apple M2 Pro chip, which includes a 16-core Neural Engine, and 16 GB of unified memory. Training, optimization, and testing of the classification model were performed in Python (3.8), using the Keras (2.4), and TensorFlow (2.3) libraries.

### H. Performance Measures

To evaluate the performance in FOG detection at window-level of the proposed framework and those reproduced from the state-of-the-art approaches, commonly used metrics were calculated and reported.

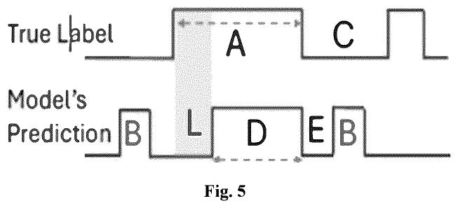

In this binary classification problem (FoG or non-FoG), performance metrics include sensitivity, specificity, F1 score, and AUC of receiver operating characteristics. LIFT-PD was evaluated using these metrics, comparable to other state-of-the-art methods. True positives (TP) are correctly identified FoG windows, while false positives (FP) are non-FoG windows incorrectly labeled as FoG. False negatives (FN) are real FOG windows not recognized, and true negatives (TN) are correctly classified non-FoG instances. FIG. 5 schematically describes these metrics. FIG. 5 illustrates A: FoG Episode, B: False Positive, C: Normal Episode, D: Detected FoG Episode (TP), E: Detected Normal Episode (TN), L: Latency. Sensitivity/Recall measures the ratio of correctly detected FOG windows and specificity measures the ratio of correctly identified non-FoG windows. Precision evaluates the model's ability to avoid false positives. The F1 score is the harmonic mean of sensitivity and precision which is used to assess performance on the imbalanced dataset [25].

### I. FoG Detection Post-Processing

For further evaluation of the LIFT-PD framework, a postprocessing analysis was performed using predictions and class labels. The performance of true FoG episode (TFE) detection was performed by analyzing groups of consecutive windows with the presence of freezing episodes, besides the window-level FoG detection.
1) FoG Episode: LIFT-PD's detection of FoG episodes—defined as consecutive windows labeled as FoG—was assessed by computing both the percentage of episodes detected and the proportion of FoG accurately identified within each episode. An episode is considered detected if at least one window within it is correctly identified. FoG episodes were categorized into three groups: short (<6 seconds), medium (6-12 seconds), and long (>12 seconds).

The percentage of FoG episodes were detected across the entire dataset and for each duration group. This analysis measured the proportion of accurately detected FoG windows within each episode (FIG. 5D), defined as

$${D}_{FoG} ( \% ) = \frac{{n}_{detected}}{{n}_{total}} ,$$

where n_{detected} represents the number of detected FoG windows, and nₜₒₜₐₗ is the total FoG windows in that episode.

For false FoG episodes, the minimum distance between each falsely detected episode (FIG. 5B) and the nearest true FoG window (FIG. 5A) was calculated to understand the proximity of false positives to actual FoG occurrences. Finally, FoG detection latency, defined as the time difference between the onset of an actual FoG episode and the detected FoG episode (FIG. 5L) was evaluated. This metric reflects the algorithm's responsiveness in identifying FoG events, which is crucial for the timely intervention and management of PD patients.

TABLE IV
Performance of proposed LIFT-PD framework.

| Group | Prec | Rec/Sens/DFW | F1 Score | Acc. | Spec. | Loss | DFE | DFW |
|---|---|---|---|---|---|---|---|---|
| 1 | 066 (0.61) | 0.82 (0.82) | 0.73 (0.70) | 0.81 (0.77) | 0.81 (0.75) | 0.19 (0.23) | 86.65% (86.7%) | 81.6% (82.0%) |
| 2 | 0.82 (0.77) | 0.86 (0.86) | 0.84 (0.81) | 0.84 (0.82) | 0.83 (0.78) | 0.16 (0.19) | 89.35% (90.1%) | 86.1% (86.1%) |
| Avg | 0.74 (0.69) | 0.84 (0.84) | 0.79 (0.76) | 0.825 (0.79) | 0.82 (0.77) | 0.18 (0.21) | 88.00% (88.4%) | 83.85% (84.1%) |
| Min | 0.66 (0.61) | 0.82 (0.82) | 0.73 (0.70) | 0.81 (0.77) | 0.81 (0.75) | 0.16 (0.19) | 86.65% (86.7%) | 81.60% (82.01%) |
| Max | 0.82 (0.77) | 0.86 (0.86) | 0.84 (0.81) | 0.84 (0.82) | 0.83 (0.78) | 0.19 (0.23) | 89.35% (90.1%) | 86.10% (86.1%) |
| STD | 0.11 (0.11) | 0.03 (0.03) | 0.08 (0.08) | 0.02 (0.03) | 0.01 (0.02) | 0.02 (0.03) | 1.91 (2.4) | 3.18 (2.9) |

Key Metrics Defined: PREC: Precision, REC: Recall, SENS: Sensitivity, ACC: Accuracy, SPEC: Specificity, DFE: Detected FoG Episodes, DFW: Detected FoG Windows, STD: Standard Deviation. Performance metrics for the baseline supervised model are presented within parentheses ( ).

2) Computational Complexity: To evaluate the feasibility of deploying the LIFT-PD framework on low-resource wearable devices, several analyses were performed. This analysis assessed training and testing times for different input sizes to identify the optimal training size for the self-supervised pre-text task using varying amounts of unlabeled data. Post-training, inference times across different input sizes were measured to gauge real-time performance.

Memory requirements for storing input sensor data and model parameters were also assessed, ensuring suitability for resource-constrained devices. To optimize power consumption and enable long-term in-home monitoring, the LIFT-PD framework implements an Activity Threshold Optimization (ATO) algorithm (Algorithm 1), activating the FoG detection model only during active periods. Assuming that the performance function P and the computation of active windows N_{α} can be performed in constant time, the overall runtime excluding the inference model is

$$O ( N \cdot \frac{{α}_{max}}{δα} ) .$$

Including the interference model's runtime O(M), where M is the process time of a single active window, the adjusted complexity becomes

$$O ( ( M + N ) \cdot \frac{{α}_{max}}{δα} ) .$$

In the best-case scenario, where the optimal threshold αₒₚₜ is found in the first iteration, the runtime is O(N).

### IV. Results

### A. Performance Analysis

The performance of the LIFT-PD framework was analyzed using mainly sensitivity, and specificity along with some other matrices (percentage of detected episode, latency, precision, F1 score, and AUC of the ROC curve), comparing it against a baseline supervised model with the same architecture and parameters. This comprehensive evaluation highlights the benefits and potential limitations of the self-supervised learning (SSL) approach for real-time FoG detection.

Table IV summarizes the metrics, showing that the SSL model achieved notable improvements: a 7.25% increase in average precision, 4.4% in accuracy, and 6.5% in specificity compared to the baseline supervised model. Importantly, the SSL model maintained consistent recall/sensitivity (84%) with the baseline, ensuring that the detection of FoG episodes was not compromised despite reduced supervision. The F1 score, which balances precision and recall, was higher by about 3.95% in the SSL model, indicating better overall performance in detecting FoG episodes.

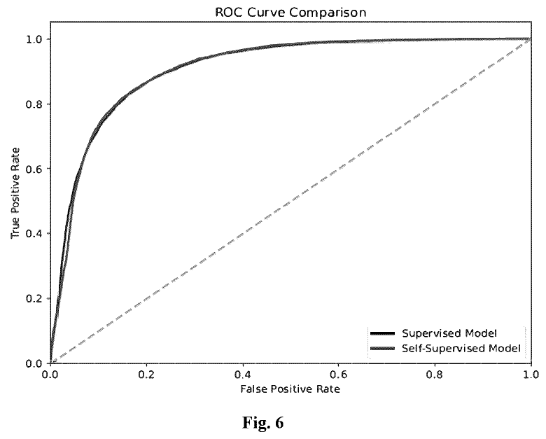

Group 2 outperformed Group 1 in all metrics. Group 1's lower precision (0.66) was due to more false positives from a higher proportion of FoG events. The DHW technique with SSL mitigated data imbalance, enhancing performance despite class imbalances in Group 1. The ROC curves in FIG. 6 showed that the SSL model had a slightly larger area under the curve (0.908) than the supervised model (0.9078), indicating better FoG classification performance. The close proximity of the AUC values between the two models highlights the robustness of the SSL approach in achieving comparable performance to the supervised baseline, despite leveraging limited labeled data during training.

FIG. 6 illustrates Receiver Operating Characteristic (ROC) curves for supervised and self-supervised models.

The performance of the self-supervised learning (SSL) approach was compared with a semi-supervised learning (Semi-SL) method for FoG detection. To isolate the value of the representation-learning strategy, this analysis swapped out the SSL stage in LIFT-PD for a II-model style semi-supervised pipeline [26], leaving the backbone network, DHWT segmentation and opportunistic inference module unchanged, and trained both variants on the identical data split. The semisupervised baseline indeed benefits from adding unlabeled examples, but in practice it needs a comparatively large seed of reliable labels (≈65-75% of the training set, vs. 40% for LIFT-PD) before its pseudo-labels stabilise. Because FoG episodes are sparse and highly variable, early pseudo-labels are often noisy; that noise propagates through the student-teacher updates and depresses recall and F1. In contrast, the self-supervised pre-text task described herein reconstructs masked sensor segments, allowing the encoder to learn gait-level structure from all recordings without ever relying on tentative class labels. The richer, label-agnostic features transfer cleanly to the small fine-tuning set, so LIFT-PD attains higher recall (0.84 vs 0.77) and f1 (0.79 vs 0.75) while matching or exceeding the baseline on precision, accuracy and specificity (Table VI).

TABLE V
Performance comparison across diverse groups.

| Group | Test Group | Pre | Rec | F1 | Spec | Acc |
|---|---|---|---|---|---|---|
| Sevirity | Severe | 0.71 (0.7) | 0.81 (0.79) | 0.75 (0.74) | 0.79 (0.79) | 0.8 (0.79) |
| (40) | Mild (14) | 0.78 (0.78) | 0.81 (0.81) | 0.80 (0.79) | 0.85 (0.84) | 0.83 (0.83) |
| Gender | Female | 0.78 (0.67) | 0.75 (0.77) | 0.76 (0.72) | 0.84 (0.71) | 0.80 (0.77) |
| (40) | Male (32) | 0.71 (0.74) | 0.67 (0.64) | 0.69 (0.68) | 0.83 (0.86) | 0.77 (0.77) |
| Age | Old (20) | 0.80 (0.75) | 0.71 (0.71) | 0.75 (0.74) | 0.86 (0.82) | 0.80 (0.78) |
| (40) | Mid-Age | 0.70 (0.66) | 0.80 (0.79) | 0.74 (0.72) | 0.82 (0.79) | 0.81 (0.79) |
| Medication | On | 0.82 (0.78) | 0.79 (0.72) | 0.81 (0.75) | 0.84 (0.82) | 0.86 (0.81) |
| | Off | 0.78 (0.70) | 0.8 (0.74) | 0.79 (0.72) | 0.79 (0.72) | 0.82 (0.78) |
| Random | 1 (20) | 0.66 (0.61) | 0.82 (0.82) | 0.73 (0.70) | 0.81 (0.75) | 0.81 (0.77) |
| (40) | 2 (20) | 0.82 (0.77) | 0.86 (0.86) | 0.84 (0.81) | 0.83 (0.78) | 0.84 (0.82) |

Performance metrics for the baseline supervised model are presented within parentheses ( ).

TABLE VI
Performance Comparison Between Self-Supervised Learning (SSL)
and Semi-Supervised Learning (Semi-SL). Performance metrics
for the Semi-SL are presented within parentheses ( ).

| Group | Prec | | Rec/Sens/DFW | F1 Score | Acc. | Spec. |
|---|---|---|---|---|---|---|
| 1 | 0.66 | (0.66) | 0.82 (0.73) | 0.73 (0.70) | 0.81 (0.80) | 0.81 (0.84) |
| 2 | 0.82 | (0.8) | 0.86 (0.81) | 0.84 (0.80) | 0.84 (0.82) | 0.83 (0.82) |
| Avg. | 0.74 | (0.74) | 0.84 (0.77) | 0.79 (0.75) | 0.83 (0.81) | 0.82 (0.83) |

% of Detected FoG Episode (DFE)

| Small, 0-6 s | Medium, 6-12 s | Large, >12 |
|---|---|---|
| 83.3% (78.56%) | 100% (96.83%) | 100% (99.23%) |

TABLE VII
Performance comparison of DHWT across datasets.

| | | | | ↑ | | |
|---|---|---|---|---|---|---|
| Dataset | Train Time | Test Time | Pre | F1 | Spec | Acc |
| tdcs | 1.8 s | 0.94 s | 0.74 | 0.8 | 0.82 | 0.83 |
| | ↑88% | (0.92 s) | ↑7.2% | ↑5.3% | ↑6.5% | ↑5.1% |
| Daphnet | 0.14 | 0.134 s | 0.55 | 0.44 | 0.94 | 0.87 |
| | ↑42% | (0.132 s) | ↑161% | ↑42% | ↑108% | ↑71% |
| MotionSense | 0.08 | 0.022 s | 1 | 0.99 | 1 | 0.99 |
| | ↑142% | (0.024 s) | ↑3.1% | ↑2.5% | ↑1.2% | ↑1.22% |

The second row shows relative improvement over the baseline.

1) Generalizability of DHWT: The analysis was expanded to Daphnet [27] and two activities from MotionSense [28](as a binary classification), in addition to the primary tdcs dataset. The results show an increase in training time across the datasets due to class imbalance handling, while the inference time remained stable. DHWT significantly improved precision, F1-score, and specificity in datasets with class imbalance, confirming its effectiveness across diverse conditions. Daphnet showed a 161% boost in precision and a 108% improvement in specificity. These results support the generalizability of DHWT without compromising computational efficiency (Table VII).
2) Robustness Across Diverse Subjects: To ensure generalizability, the dataset (N=40) was split into diverse groups based on key characteristics such as severity (Mild N=14), gender (Male N=32) and age (Mid-age N=20). These groups were deliberately formed to include outliers and reflect real-world diversity, ensuring that the model can handle a variety of subject profiles without overfitting to any particular population. Improvements in F1 scores, accuracy and specificity were observed in all groups, particularly in mild cases and older age groups, confirming that the method works effectively even with varied patient characteristics.

Regarding the medication status, the model was evaluated separately on data from patients in their “On” and “Off” medication states. Patients in the “On” state generally show fewer motor symptoms due to the effects of dopaminergic treatment, while the “Off” state is marked by more pronounced motor symptoms, including fluctuations. Interestingly, when both “On” and “Off” states were combined during training, the model demonstrated superior results by better capturing the intra-patient variability between medicated and unmedicated states (Random group-2). This suggests that LIFT-PD can adapt to fluctuating motor symptoms effectively, providing accurate detection across different medication states, which is critical for real-world clinical monitoring and intervention planning.

### B. Clinical Correlation of Predicted FoG Scores

To assess the clinical relevance of LIFT-PD's predictions, correlation coefficients were computed between the model's FoG scores and standard clinical metrics. Here, FoG scores were defined as the per-subject proportion of detected FoG windows during test sessions. This metric provides a summary-level indicator of how frequently the model detected FoG-related patterns in each participant. It was found that the FoG scores were significantly correlated with NFOGQ scores (Pearson's r=0.72, p<0.01) and UPDRS-III Off scores (r=0.63, p<0.05), both widely used clinical markers of disease severity and motor impairment. These results indicate strong alignment between the model's predictions and clinical assessments, supporting the model's translational potential for patient monitoring.

### C. Statistical Analysis of Performance Differences

TABLE VIII
The paired t-test results comparing
LIFT-PD to the supervised models

| | t- | p- | |
|---|---|---|---|
| Metric | statistic | value | Interpretation |
| Precision | 3.18 | 0.011 | Statistically significant improvement |
| | | | with LIFT-PD (p < 0.05) |
| Recall | 1.87 | 0.094 | Not statistically significant (p > 0.05), |
| | | | though LIFT-PD shows a positive trend |
| F1 Score | 4.20 | 0.0023 | Highly significant improvement in F1 |
| | | | score with LIFT-PD. |
| Specificity | 2.74 | 0.023 | Significant improvement in reducing false |
| | | | positives (p < 0.05). |
| Accuracy | 4.27 | 0.0021 | Highly significant improvement in overall |
| | | | accuracy |

To statistically validate the improvements observed with LIFT-PD over the supervised baseline, paired t-tests were conducted across multiple performance metrics. The results show statistically significant improvements in precision (p=0.011), F1-score (p=0.0023), specificity (p=0.023), and accuracy (p=0.0021), confirming that LIFT-PD outperforms the supervised model in key areas relevant to clinical application. Although the improvement in recall (p=0.094) was not statistically significant at the 5% level, it still showed a positive trend, suggesting consistent performance across diverse patient groups. These findings support the robustness and reliability of the proposed approach.

D. Compare with State-of-the-Art Models

Table IX compares the performance of the LIFT-PD model with state-of-the-art methods for detecting FoG in PD patients. For fairness, all models were implemented from scratch and evaluated on the same dataset using consistent experimental protocols. Although the Multi-head CNN [12] achieves the highest detection rates for FoG episodes (97.27%) and windows (94.64%), its precision (0.545) and specificity (0.491) are low, indicating a high false positive rate. High false positives can reduce the effectiveness of cueing due to patients' adaptiveness to “always on” interventions [29][30]. The one-class classifier [10] shows high precision (0.856) and specificity (0.891) but lower recall (0.716), missing many true FoG events, which can lead to inadequate monitoring and delayed interventions. The semi-supervised model [11] shows a recall around 12% lower and specificity approximately 13% lower than LIFT-PD, making it less suitable for in-home monitoring with a single accelerometer.

The LIFT-PD model achieves a balanced performance with 88% episode detection, 83.85% window detection, 0.74 precision, 0.84 recall, 0.79 F1-score, and 0.82 specificity. These results show that LIFT-PD achieves comparable performance to the supervised methods while being more suitable for real-time wearable deployment with limited computational resources for remote monitoring.

TABLE IX
Comparison with state-of-the-art models

| | | Rec/Sens/ | | F1 | |
|---|---|---|---|---|---|
| Study | DFE | DFW | Prec | Score | Spec. |
| One Class Classifier [10] | 90.8% | 71.6% | 0.86 | 0.77 | 0.89 |
| Semi-Supervised | — | 72.3% | — | — | 0.71 |
| Model [11] | | | | | |
| Multi-head CNN [12] | 97.3% | 94.6% | 0.56 | 0.68 | 0.49 |
| LIFT-PD | 88% | 84% | 0.74 | 0.79 | 0.82 |

### E. Duration Based FoG Episode

Table X presents the detection rates for FoG episodes and windows, along with associated latency metrics, grouped into short, medium, and long durations. The SSL model's performance is compared with a baseline supervised model (metrics in parentheses).

The SSL model shows robust performance across all durations, with detection rates increasing as the episode lengthens. This trend is mirrored in the baseline model's performance, indicating a consistent improvement across different modeling approaches. For short episodes, the detection rate is 83.3% for episodes and 68% for windows, with an average latency of 2.42±0.45 seconds. For long episodes, these rates rise to 100% for episodes and 91.1% for windows, with a latency of 2.64±1.45 seconds. The increasing detection rates for longer episodes are due to the more prominent and persistent FoG characteristics, such as tremors and shuffling gait, which the SSL model effectively captures. The increasing detection rates for longer episodes are due to the more prominent and persistent FoG characteristics, such as tremors and shuffling gait, which LIFT-PD effectively captures.

TABLE X
Duration-based FoG episode analysis.

| | FoG Episodes & windows detection rate (%) | | | | |
|---|---|---|---|---|---|
| | with Latency (s) and standard deviation | | | | |
| Duration | Small, 0-6 s | | Medium, 6-12 s | | Large, >12 s |
| FoG Episodes | 83.3% | (82.8%) | 100% | (98.8%) | 100% (100%) |
| FoG Windows | 68% | (71.4%) | 81.9% | (84.1%) | 91.1% (92.6%) |

| | | | |
|---|---|---|---|
| Avg. | 2.42 ± 0.45 s | 2.6 ± 0.96 s | 2.64 ± 1.45 s |
| Latency ± SD | (2.38 ± 0.45 s) | (2.5 ± 0.82 s) | (2.6 ± 1.29 s) |
| Max. Latency | 4.5 s | 6 s | 9.75 s |
| | (4.5 s) | (5.25 s) | (9.75 s) |

Latency, representing the detection delay from episode onset, slightly increases with duration. Small episodes have an average latency of 2.42±0.45 seconds, while long episodes reach 2.64±1.45 seconds. Maximum latency values increase from 4.5 seconds for small episodes to 9.75 seconds for long ones, reflecting the need to analyze more data over time to confidently detect FoG onset. Despite this latency increase, the detection accuracy for longer episodes remains superior, showing the SSL model's effectiveness in real-world applications by balancing latency and accuracy for reliable FoG detection across varying episode durations.

### F. Threshold Effect in MAM

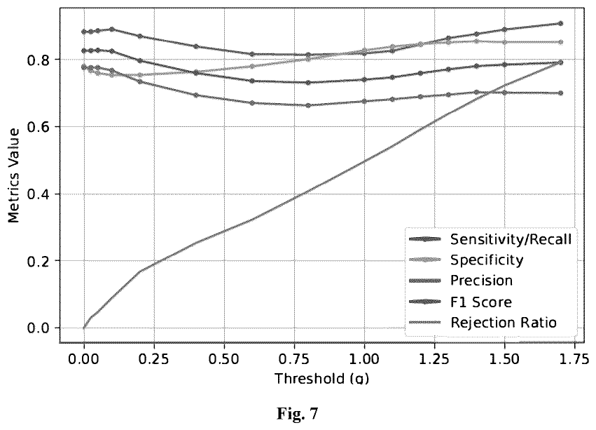

To determine the optimal activity threshold for the MAM, the impact of different threshold values on various performance metrics were evaluated, as presented in Table XI and FIG. 7. As observed in Table XI, lowering the activity threshold (e.g., 0.0) results in higher sensitivity (0.884) and higher detected FoG episode (DFE) rate (0.92), indicating that more FoG events are detected. However, this comes at the cost of lower specificity (0.78) and a higher inference time (3.31 ms) due to the SSL model being activated more frequently, even during inactive periods. Conversely, increasing the activity threshold (e.g., 1.2) leads to higher specificity (0.846) and a lower rejection ratio (0.59), implying that fewer false positive detections occur during inactive periods. However, this improvement is accompanied by a slight decrease in sensitivity (0.845), DFE rate (0.809), and an increase in the number of missed FoG events.

TABLE XI
Effect of different activation thresholds on MAM

| Threshold | Sens | Spec | DFE | Rejection Ratio | Inference Time | |
|---|---|---|---|---|---|---|
| 0 | 0.884 | 0.78 | 0.92 | 0 | 3.31 | ms |
| 0.2 | 0.87 | 0.75 | 0.92 | 0.17 | 1.96 | ms |
| 0.4 | 0.839 | 0.764 | 0.896 | 0.25 | 1.76 | ms |
| 1.2 | 0.845 | 0.846 | 0.809 | 0.59 | 1.1 | ms |

FIG. 7 illustrates the Impact of activity threshold on SSL performance.

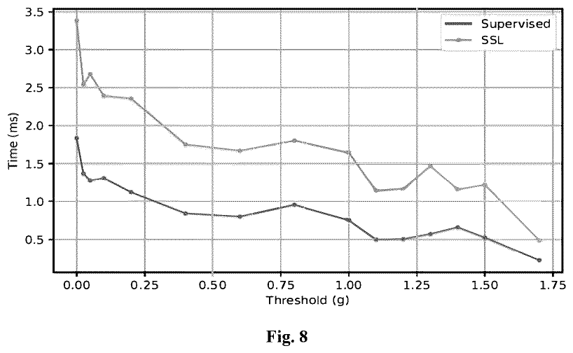

FIG. 8 illustrates the Effect of activity threshold on inference time.

FIG. 7 shows how metrics like specificity and rejection ratio increase steadily with the threshold, while sensitivity and overall detection performance (F1 score) gradually decline. Careful selection of the threshold is crucial to balance the requirements of accurately detecting active periods while effectively rejecting inactive intervals to optimize computational resources. FIG. 8 shows that as the activity threshold increases, the model execution time decreases for both supervised and self-supervised models. At a threshold of 1.2 g, the SSL model's execution time reaches approximately 1.1 ms (Table XI, FIG. 8). This reduction is due to the MAM effectively filtering out inactive periods, reducing the need for the computationally intensive SSL model. The SSL model has slightly higher inference times than the supervised model due to an additional MLP layer on top of the pre-trained encoder. Despite the modest increase, the SSL model demonstrates enhanced robustness and superior performance using only 40-60% of labeled training data. The average execution time per window is 0.0295 ms for the supervised model and 0.0379 ms for the SSL model.

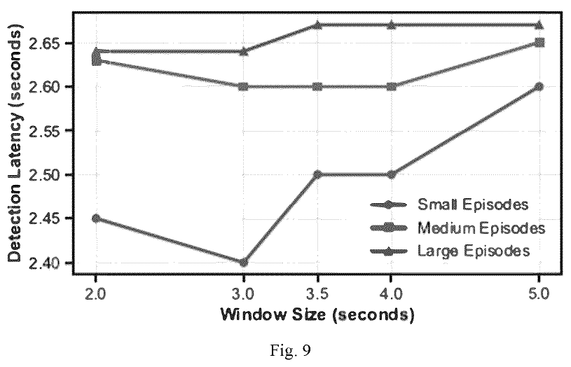

FIG. 9 illustrates the Impact of window size on FoG detection latency across different episode durations (Small, Medium, Large).

### G. Effect of Window Size in Episode Detection

Extensive validation was conducted by randomly taking a 10% subset of the training data as a validation set. The results indicated that longer windows, such as 4 s or 5 s, showed a delay in detecting FoG episodes, possibly due to including unnecessary gait segments not closely linked to FoG onset, in line with previous studies [17], [31]. Among evaluated window lengths (2-5 seconds), the 3-second window was optimal, balancing detection latency and data relevance. Although intuitively shorter windows (2s) could reduce latency in some cases for larger FoG episodes, the validation analysis described herein revealed that shorter windows contained insufficient contextual information for reliable detection, thus slightly increasing the average detection latency compared to the 3-second window shown in FIG. 9. Longer windows (3.5 s, 4 s, 5 s) further increased latency without accuracy gains. Therefore, a 3-second window was selected for optimal performance.

### H. Effect of Labels During Pre-Training

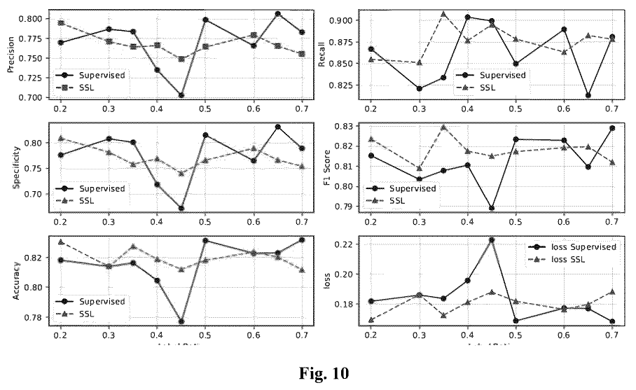

FIG. 10 illustrates how varying the label ratio (on the x-axis) impacts the performance (on the y-axis) of the self-supervised learning (SSL) and supervised models during training.

As the ratio increases from 0.2 to 0.7, (meaning more labeled data is available for training), both models generally show an upward trend in metrics such as precision, recall, F1-score, and accuracy, which aligns with the expected behavior when more labeled data is available. However, a notable observation was the SSL model's superior stability and consistency compared to the supervised counterpart, which displayed sharper performance fluctuations across different label ratios.

FIG. 10: Performance analysis for different amounts of labeled data.

This steadiness underscores the SSL model's robustness and reduced sensitivity to the availability of labeled data during training. It also highlights the model's capability to leverage pre-training on unlabeled data to learn generalized features transferable to the target task. FIG. 10 also shows that SSL model not only competes closely but sometimes outperforms the supervised model, especially with 40-60% labeled data. This indicates that the SSL model can achieve promising results with fewer labeled instances, making it highly efficient and adaptable for scenarios where obtaining large amounts of labeled data is challenging.

### I. Post Processing

The post-processing analysis evaluated the temporal dynamics between false positives (FPs) and true FoG episodes (TFE) detected by the LIFT-PD framework. On average, FPs occurred 16 seconds after the previous TFE and 18.5 seconds before the next true episode, suggesting they often arise from residual motor instability or sensor inaccuracies immediately following an actual FoG event. A ‘Pre FP Analysis’ shows that FPs were found to occur approximately 14 seconds away from the nearest TFE, indicating the system's high sensitivity to subtle motor pattern changes preceding or following a FoG episode. These false alarms could serve as precursors or warnings of impending FoG events, providing valuable time windows for treatment adjustments. To address isolated false detections, a majority voting scheme was implemented. If a window's classification differed from its immediate neighbors, it was adjusted to match them, smoothing the detection sequence. This approach improved the detection performance, increasing the detected true FoG episodes from 88% to 89.8% and true FoG windows from 83.85% to 84.64%.

### V. Limitations

While LIFT-PD demonstrates promising results and practical advantages for real-world deployment, several limitations remain. First, although the model generalizes well across diverse datasets and patient groups, further validation in larger patient cohorts with varying clinical conditions and disease progression stages, including patients exhibiting tremor-dominant or other atypical Parkinsonian motor symptoms, is needed to confirm robustness and clinical applicability. Specifically, additional assessment is required to determine the model's accuracy in detecting FoG episodes in the presence of confounding symptoms such as resting tremors, dyskinesia, or bradykinesia, which often coexist in individuals with Parkinson's disease. Second, while the system operates effectively with a single accelerometer, incorporating additional sensor modalities such as gyroscopes or physiological sensors (i.e., heart rate, skin conductance) might enhance detection accuracy, particularly for subtle or early-stage FoG events. Third, the Opportunistic Inference Module (OIM), though significantly improving energy efficiency, might occasionally delay activation, potentially missing very brief or subtle FoG events. Fourth, although decision thresholds in OIM were empirically optimized using a small validation subset from the training data, this threshold selection process remains dataset-specific and may not generalize across varying conditions or sensor configurations. Free-living environments introduce high variability; therefor dataset-agnostic calibration techniques are being assessed using an independent dataset under acquisition. This future work aims to improve the robustness of threshold selection and ensure consistent performance across diverse deployment settings. Finally, despite using significantly fewer labeled samples through self-supervised learning, the initial tuning of parameters and training still relies partially on labeled data, and completely unsupervised adaptation remains an open challenge. Future work should address these aspects to further optimize performance, clinical relevance, and adaptability of LIFT-PD.

### VI. Discussion

LIFT-PD is a computationally efficient and robust self-supervised learning framework designed specifically for real-time, patient-independent detection of Freezing of Gait (FoG) episodes in patients with Parkinson's Disease (PD). The proposed approach addresses key challenges inherent in continuous in-home FoG monitoring, particularly the reliance on large, extensively labeled datasets and substantial power consumption. By implementing a novel Differential Hopping Windowing Technique (DHWT), LIFTPD effectively handles imbalanced data and diverse gait variations, thereby reducing annotation burdens and enhancing clinical scalability. Furthermore, the integration of an opportunistic inference module substantially decreases power consumption by activating the deep learning model exclusively during ambulatory periods. Such selective activation translates to a significant improvement in battery life, making continuous (>48-hour) wearable monitoring practical. This system requires minimal data preprocessing, utilizing only a single triaxial accelerometer placed comfortably at the waist—an approach traditionally considered suboptimal yet proven highly effective through a self-supervised learning strategy. Clinically, accurate and real-time FoG detection as provided by LIFT-PD enables timely cue delivery (e.g., rhythmic auditory, visual, or vibrotactile stimuli), critical for breaking or preventing episodes without causing habituation [3], [32]. To evaluate the clinical robustness of model described herein, performance was stratified by disease severity, age, gender, and medication state. These subgroups reflect known sources of variability in Parkinson's Disease presentation and management. LIFT-PD consistently achieved high F1 scores and specificity across all groups, with particularly strong performance in older adults and those in the On-medication state. The system's ability to robustly detect FoG episodes across varying patient demographics-including different severities, medication states, and age groups-further emphasizes its practical applicability for real-world clinical scenarios, enabling more personalized patient management.

### VII. Conclusion

In conclusion, LIFT-PD presents a practical and clinically meaningful solution for real-time detection of Freezing of Gait episodes in Parkinson's Disease. The system successfully achieves robust, patient-independent monitoring through innovative self-supervised learning and opportunistic inference strategies, reducing dependency on labeled data and significantly improving battery life. Its reliance on a single waist-worn accelerometer ensures patient comfort and adherence, thus enhancing feasibility for continuous at-home use. Furthermore, LIFT-PD demonstrates robust generalization across age, gender, disease severity, and medication states, and its outputs show significant correlation with clinical assessments (e.g., NFOGQ and UPDRS-III Off scores), reinforcing its translational potential. Ultimately, by delivering accurate and timely FoG detection and supporting targeted, personalized cue-based interventions, LIFT-PD significantly advances in-home monitoring capabilities, improves PD symptom management, and contributes positively to patients' quality of life.

Under an additional embodiment a trained LIFT-PD classification model may be transmitted to a mobile device of a user. The classification model then runs as an application on a processor of the mobile device. The classification is communicatively coupled to a wearable device of a user, e.g. a accelerometer. Under the embodiment, the classification model monitors the accelerometer data and applies the classification to detect freeze of gate events and alert the mobile device user. The classification model operates in a manner described above from pre-processing to classification. This includes activating the classification model only when a magnitude of the accelerometer data exceeds a threshold value. Further, the classification model (or application) is communicatively coupled to applications running on remote servers. In the event freeze of gait is detected, information of the event may be transmitted to one or more remote server applications which may then forward the information to remote computing devices. Therefore, third parties may be alerted to freeze of gate events through devices communicatively coupled to the remoted server.

Computer networks suitable for use with the embodiments described herein include local area networks (LAN), wide area networks (WAN), Internet, or other connection services and network variations such as the world wide web, the public internet, a private internet, a private computer network, a public network, a mobile network, a cellular network, a value-added network, and the like. Computing devices coupled or connected to the network may be any microprocessor controlled device that permits access to the network, including terminal devices, such as personal computers, workstations, servers, mini computers, main-frame computers, laptop computers, mobile computers, palm top computers, hand held computers, mobile phones, TV set-top boxes, or combinations thereof. The computer network may include one of more LANs, WANs, Internets, and computers. The computers may serve as servers, clients, or a combination thereof.

The Self-Supervised Learning and Opportunistic Inference for Continuous Monitoring of Field of Gate in Parkinson's Disease can be a component of a single system, multiple systems, and/or geographically separate systems. The Self-Supervised Learning and Opportunistic Inference for Continuous Monitoring of Field of Gate in Parkinson's Disease can also be a subcomponent or subsystem of a single system, multiple systems, and/or geographically separate systems. The components of Self-Supervised Learning and Opportunistic Inference for Continuous Monitoring of Field of Gate in Parkinson's Disease can be coupled to one or more other components (not shown) of a host system or a system coupled to the host system.

One or more components of the Self-Supervised Learning and Opportunistic Inference for Continuous Monitoring of Field of Gate in Parkinson's Disease and/or a corresponding interface, system or application to which the Self-Supervised Learning and Opportunistic Inference for Continuous Monitoring of Field of Gate in Parkinson's Disease is coupled or connected includes and/or runs under and/or in association with a processing system. The processing system includes any collection of processor-based devices or computing devices operating together, or components of processing systems or devices, as is known in the art. For example, the processing system can include one or more of a portable computer, portable communication device operating in a communication network, and/or a network server. The portable computer can be any of a number and/or combination of devices selected from among personal computers, personal digital assistants, portable computing devices, and portable communication devices, but is not so limited. The processing system can include components within a larger computer system.

The processing system of an embodiment includes at least one processor and at least one memory device or subsystem. The processing system can also include or be coupled to at least one database. The term “processor” as generally used herein refers to any logic processing unit, such as one or more central processing units (CPUs), digital signal processors (DSPs), application-specific integrated circuits (ASIC), etc. The processor and memory can be monolithically integrated onto a single chip, distributed among a number of chips or components, and/or provided by some combination of algorithms. The methods described herein can be implemented in one or more of software algorithm(s), programs, firmware, hardware, components, circuitry, in any combination.

The components of any system that include the Self-Supervised Learning and Opportunistic Inference for Continuous Monitoring of Field of Gate in Parkinson's Disease can be located together or in separate locations. Communication paths couple the components and include any medium for communicating or transferring files among the components. The communication paths include wireless connections, wired connections, and hybrid wireless/wired connections. The communication paths also include couplings or connections to networks including local area networks (LANs), metropolitan area networks (MANs), wide area networks (WANs), proprietary networks, interoffice or backend networks, and the Internet. Furthermore, the communication paths include removable fixed mediums like floppy disks, hard disk drives, and CD-ROM disks, as well as flash RAM, Universal Serial Bus (USB) connections, RS-232 connections, telephone lines, buses, and electronic mail messages.

Aspects of the Self-Supervised Learning and Opportunistic Inference for Continuous Monitoring of Field of Gate in Parkinson's Disease and corresponding systems and methods described herein may be implemented as functionality programmed into any of a variety of circuitry, including programmable logic devices (PLDs), such as field programmable gate arrays (FPGAs), programmable array logic (PAL) devices, electrically programmable logic and memory devices and standard cell-based devices, as well as application specific integrated circuits (ASICs). Some other possibilities for implementing aspects of the Self-Supervised Learning and Opportunistic Inference for Continuous Monitoring of Field of Gate in Parkinson's Disease and corresponding systems and methods include: microcontrollers with memory (such as electronically erasable programmable read only memory (EEPROM)), embedded microprocessors, firmware, software, etc. Furthermore, aspects of the Self-Supervised Learning and Opportunistic Inference for Continuous Monitoring of Field of Gate in Parkinson's Disease and corresponding systems and methods may be embodied in microprocessors having software-based circuit emulation, discrete logic (sequential and combinatorial), custom devices, fuzzy (neural) logic, quantum devices, and hybrids of any of the above device types. Of course the underlying device technologies may be provided in a variety of component types, e.g., metal-oxide semiconductor field-effect transistor (MOSFET) technologies like complementary metal-oxide semiconductor (CMOS), bipolar technologies like emitter-coupled logic (ECL), polymer technologies (e.g., silicon-conjugated polymer and metal-conjugated polymer-metal structures), mixed analog and digital, etc.

It should be noted that any system, method, and/or other components disclosed herein may be described using computer aided design tools and expressed (or represented), as data and/or instructions embodied in various computer-readable media, in terms of their behavioral, register transfer, logic component, transistor, layout geometries, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, etc.). When received within a computer system via one or more computer-readable media, such data and/or instruction-based expressions of the above described components may be processed by a processing entity (e.g., one or more processors) within the computer system in conjunction with execution of one or more other computer programs.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import, when used in this application, refer to this application as a whole and not to any particular portions of this application. When the word “or” is used in reference to a list of two or more items, that word covers all of the following interpretations of the word: any of the items in the list, all of the items in the list and any combination of the items in the list.

The above description of embodiments of the Self-Supervised Learning and Opportunistic Inference for Continuous Monitoring of Field of Gate in Parkinson's Disease is not intended to be exhaustive or to limit the systems and methods to the precise forms disclosed. While specific embodiments of, and examples for, the Self-Supervised Learning and Opportunistic Inference for Continuous Monitoring of Field of Gate in Parkinson's Disease and corresponding systems and methods are described herein for illustrative purposes, various equivalent modifications are possible within the scope of the systems and methods, as those skilled in the relevant art will recognize. The teachings of the Self-Supervised Learning and Opportunistic Inference for Continuous Monitoring of Field of Gate in Parkinson's Disease and corresponding systems and methods provided herein can be applied to other systems and methods, not only for the systems and methods described above.

The elements and acts of the various embodiments described above can be combined to provide further embodiments. These and other changes can be made to the Self-Supervised Learning and Opportunistic Inference for Continuous Monitoring of Field of Gate in Parkinson's Disease and corresponding systems and methods in light of the above detailed description.

### REFERENCES

[1]A. Nieuwboer and N. Giladi, “Characterizing freezing of gait in parkinson's disease: models of an episodic phenomenon,” Movement Disorders, vol. 28, no. 11, pp. 1509-1519, 2013.
[2]L.-L. Zhang, S. D. Canning, and X.-P. Wang, “Freezing of gait in parkinsonism and its potential drug treatment,” Current neuropharmacology, vol. 14, no. 4, p. 302, 2016.
[3]C. Cosentino, M. Putzolu, S. Mezzarobba, M. Cecchella, T. Innocenti, G. Bonassi, A. Botta, G. Lagravinese, L. Avanzino, and E. Pelosin, “One cue does not fit all: a systematic review with meta-analysis of the effectiveness of cueing on freezing of gait in parkinson's disease,” Neuroscience & Biobehavioral Reviews, p. 105189, 2023.
[4]P. L. Jadhwani and P. Harjpal, “A review of artificial intelligence-based gait evaluation and rehabilitation in parkinson's disease,” Cureus, vol. 15, no. 10, 2023.
[5]L. Borzi, I. Mazzetta, A. Zampogna, A. Suppa, G. Olmo, and F. Irrera, “Prediction of freezing of gait in parkinson's disease using wearables and machine learning,” Sensors, vol. 21, no. 2, p. 614, 2021.
[6]T. Bikias, D. Iakovakis, S. Hadjidimitriou, V. Charisis, and L. J. Hadjileontiadis, “Deepfog: an imu-based detection of freezing of gait episodes in parkinson's disease patients via deep learning,” Frontiers in Robotics and AI, vol. 8, p. 537384, 2021.
[7]A. L. S. de Lima, L. J. W. Evers, T. Hahn, L. Bataille, J. L. Hamilton, M. A. Little, Y. Okuma, B. R. Bloem, and M. J. Faber, “Freezing of gait and fall detection in parkinson's disease using wearable sensors: a systematic review,” Journal of Neurology, vol. 264, pp. 1642-1654, 2017. [Online]. Available: https://api.semanticscholar.org/CorpusID:3626185
[8]N. Peppes, P. Tsakanikas, E. Daskalakis, T. Alexakis, E. Adamopoulou, and K. Demestichas, “Foggan: Generating realistic parkinson's disease freezing of gait data using gans,” Sensors, vol. 23, no. 19, p. 8158, 2023.
[9]L. Sigcha, L. Borz\acute{ }i, I. Pav\acute{ }on, N. Costa, S. Costa, P. Arezes, J. M. L\acute{ }opez, and G. De Arcas, “Improvement of performance in freezing of gait detection in parkinson's disease using transformer networks and a single waist-worn triaxial accelerometer,” Engineering Applications of Artificial Intelligence, vol. 116, p. 105482, 2022.
[10]N. Naghavi and E. Wade, “Towards real-time prediction of freezing of gait in patients with parkinson's disease: a novel deep one-class classifier,” IEEE Journal of Biomedical and Health Informatics, vol. 26, no. 4, pp. 1726-1736, 2021.
[11]V. Mikos, C.-H. Heng, A. Tay, N. S. Y. Chia, K. M. L. Koh, D. M. L. Tan, and W. L. Au, “Real-time patient adaptivity for freezing of gait classification through semi-supervised neural networks,” in 2017 16th IEEE International Conference on Machine Learning and Applications (ICMLA). IEEE, 2017, pp. 871-876.
[12]L. Borzì, L. Sigcha, D. Rodriguez-Martin, and G. Olmo, “Real-time detection of freezing of gait in parkinson's disease using multi-head convolutional neural networks and a single inertial sensor,” Artif Intell. Med., vol. 135, no. C, jan 2023. [Online]. Available: https://doi.org/10.1016/j.artmed.2022.102459
[13]W. Zhang, Z. Yang, H. Li, D. Huang, L. Wang, Y. Wei, L. Zhang, L. Ma, H. Feng, J. Pan et al., “Multimodal data for the detection of freezing of gait in parkinson's disease,” Scientific data, vol. 9, no. 1, pp. 1-10, 2022.
[14]Y. Guo, D. Huang, W. Zhang, L. Wang, Y. Li, G. Olmo, Q. Wang, F. Meng, and P. Chan, “High-accuracy wearable detection of freezing of gait in parkinson's disease based on pseudo-multimodal features,” Computers in Biology and Medicine, vol. 146, p. 105629, 2022.
[15]R. R. Azghan, N. C. Glodosky, R. K. Sah, C. Cuttler, R. McLaughlin, M. J. Cleveland, and H. Ghasemzadeh, “Personalized modeling and detection of moments of cannabis use in free-living environments,” in 2023 IEEE 19th International Conference on Body Sensor Networks (BSN), 2023, pp. 1-4.
[16]J. Wang, T. Zhu, J. Gan, L. L. Chen, H. Ning, and Y. Wan, “Sensor data augmentation by resampling in contrastive learning for human activity recognition,” IEEE Sensors Journal, vol. 22, no. 23, pp. 22 994-23 008, 2022.
[17]K. Koltermann, J. Clapham, G. Blackwell, W. Jung, E. N. Burnet, Y. Gao, H. Shao, L. Cloud, I. Pretzer-Aboff, and G. Zhou, “Gaitguard: Turn-aware freezing of gait detection for non-intrusive intervention systems,” in 2024 IEEE/ACM Conference on Connected Health: Applications, Systems and Engineering Technologies (CHASE). IEEE, 2024, pp. 61-72.
[18]T. Chen, S. Kornblith, M. Norouzi, and G. Hinton, “A simple framework for contrastive learning of visual representations,” 2020.
[19]B. Khaertdinov, E. Ghaleb, and S. Asteriadis, “Contrastive selfsupervised learning for sensor-based human activity recognition,” in 2021 IEEE International Joint Conference on Biometrics (IJCB). IEEE, 2021, pp. 1-8.
[20]Z. Liu, A. Alavi, M. Li, and X. Zhang, “Self-supervised contrastive learning for medical time series: A systematic review,” Sensors, vol. 23, no. 9, p. 4221, 2023.
[21]A. Howard, A. Salomon, E. Gazit, HCL-Jevster, J. Hausdorff, L. Kirsch, Maggie, P. Ginis, R. Holbrook, and Y. F. Karim, “Parkinson's freezing of gait prediction,” Kaggle, 2023. [Online]. Available: https://kaggle.com/competitions/tlvmc-parkinsons-freezing-gait-prediction
[22]T. Reches, M. Dagan, T. Herman, E. Gazit, N. A. Gouskova, N. Giladi, B. Manor, and J. M. Hausdorff, “Using wearable sensors and machine learning to automatically detect freezing of gait during a fog-provoking test,” Sensors, vol. 20, no. 16, p. 4474, 2020.
[23]B. Manor, M. Dagan, T. Herman, N. A. Gouskova, V. G. Vanderhorst, N. Giladi, T. G. Travison, A. Pascual-Leone, L. A. Lipsitz, and J. M. Hausdorff, “Multitarget transcranial electrical stimulation for freezing of gait: a randomized controlled trial,” Movement Disorders, vol. 36, no. 11, pp. 2693-2698, 2021.
[24]K. Ziegler, F. Schroeteler, A. O. Ceballos-Baumann, and U. M. Fietzek, “A new rating instrument to assess festination and freezing gait in parkinsonian patients,” Movement Disorders, vol. 25, no. 8, pp. 1012-1018, 2010.
[25]I. R. Rahman, S. B. Soumma, and F. B. Ashraf, “Machine learning approaches to metastasis bladder and secondary pulmonary cancer classification using gene expression data,” in 2022 25th International Conference on Computer and Information Technology (ICCIT), 2022, pp. 430-435.
[26]K. Sohn, D. Berthelot, C.-L. Li, Z. Zhang, N. Carlini, E. D. Cubuk, A. Kurakin, H. Zhang, and C. Raffel, “Fixmatch: simplifying semisupervised learning with consistency and confidence,” in Proceedings of the 34th International Conference on Neural Information Processing Systems, ser. NIPS '20. Red Hook, NY, USA: Curran Associates Inc., 2020.
[27]D. Roggen, M. Plotnik, and J. Hausdorff, “Daphnet Freezing of Gait,” UCI Machine Learning Repository, 2010, DOI: https://doi.org/10.24432/C56K78.
[28]M. Malekzadeh, R. G. Clegg, A. Cavallaro, and H. Haddadi, “Mobile sensor data anonymization,” in Proceedings of the International Conference on Internet of Things Design and Implementation, ser. IoTDI '19. New York, NY, USA: ACM, 2019, pp. 49-58. [Online]. Available: http://doi.acm.org/10.1145/3302505.3310068
[29]P. Ginis, E. Nackaerts, A. Nieuwboer, and E. Heremans, “Cueing for people with parkinson's disease with freezing of gait: a narrative review of the state-of-the-art and novel perspectives,” Annals ofphysical and rehabilitation medicine, vol. 61, no. 6, pp. 407-413, 2018.
[30]E. Cubo, S. Leurgans, and C. G. Goetz, “Short-term and practice effects of metronome pacing in parkinson's disease patients with gait freezing while in the ‘on’ state: randomized single blind evaluation,” Parkinsonism & related disorders, vol. 10, no. 8, pp. 507-510, 2004.
[31]S. Mazilu, M. Hardegger, Z. Zhu, D. Roggen, G. TrOster, M. Plotnik, and J. M. Hausdorff, “Online detection of freezing of gait with smartphones and machine learning techniques,” in 2012 6th International Conference on Pervasive Computing Technologies for Healthcare (PervasiveHealth) and Workshops. IEEE, 2012, pp. 123-130.
[32]A. Nieuwboer, “Cueing for freezing of gait in patients with parkinson's disease: a rehabilitation perspective,” Movement disorders: official journal of the Movement Disorder Society, vol. 23, no. S2, pp. S475-S481, 2008.

1. A method comprising,
receiving first accelerometer data of a plurality of subjects;
processing the first accelerometer data to generate a first dataset and a second dataset, wherein the first dataset omits labeling information of freeze of gait events, wherein the second dataset includes labeling information of freeze of gait events;
training an encoder block using the first dataset;
generating a classification model using the trained encoder and the second dataset;
applying the classification model to second accelerometer data of a subject to identify a health state of the subject.

2. The method of claim 1, wherein the training the encoder block comprises masking fixed length segments of the first dataset to produce a third dataset.

3. The method of claim 2, wherein the training the encoder block comprises producing a lower dimensional representation of the third dataset.

4. The method of claim 3, wherein the training the encoder block comprises passing the lower dimensional representation through a fully connected neural network to generate encoder block weights for predicting the masked fix length segments.

5. The method of claim 1, wherein the generating the classification model comprises adding additional neural network layers to the trained encoder.

6. The method of claim 5, wherein the generating the classification model comprises training the additional layers using the second dataset and while freezing weights of the pretrained encoder.

7. The method of claim 1, wherein the applying the classification model includes monitoring a magnitude of the second accelerometer data.

8. The method of claim 7, wherein the applying the classification model comprises activating the classification model when a magnitude of the second accelerometer data exceeds a threshold value.

9. The method of claim 1, wherein the processing the first accelerometer data includes balancing freeze of gait and non freeze of gait portions of the training data.

10. The method of claim 9, wherein the balancing comprises applying a windowing overlap to freeze of gait and non freeze of gait portions of the accelerometer data.

11. The method of claim 10, wherein the windowing overlap includes a fifty percent overlap for non field of gate periods.

12. The method of claim 10, wherein the windowing overlap includes a seventy five percent overlap for field of gate periods.

13. The method of claim 1, wherein the state comprises a presence or absence of a freeze of gate event.

14. A system comprising,
one or more applications running on a server, the one or more applications for providing,
receiving first accelerometer data of a plurality of subjects;
processing the first accelerometer data to generate a first dataset and a second dataset, wherein the first dataset omits labeling information of freeze of gait events, wherein the second dataset includes labeling information of freeze of gait events;
training an encoder block using the first dataset;
generating a classification model using the trained encoder and the second dataset;
the one or more applications providing the classification model to a wearable device as a mobile application, wherein the mobile application runs on an a processor of the wearable device, wherein the wearable device monitors second accelerometer data of a wearable device user, wherein the mobile application applies the classification model to the second accelerometer data of the user to identify and notify the user of a freeze of gate event, wherein the applying the classification model includes monitoring a magnitude of the second accelerometer data and activating the classification model when a magnitude of the second accelerometer data exceeds a threshold value.

15. The method of claim 14, wherein the generating the classification model comprises adding additional neural network layers to the trained encoder.

16. The method of claim 15, wherein the generating the classification model comprises training the additional layers using the second dataset and while freezing weights of the pretrained encoder.

17. The method of claim 14, wherein the processing the first accelerometer data includes balancing freeze of gait and non freeze of gait portions of the training data.

18. The method of claim 17, wherein the balancing comprises applying a windowing overlap to freeze of gait and non freeze of gait portions of the accelerometer data.
